(19) United States
(12) Patent Application Publication
(10) Pub. No.: US 2004/0013708 A1
(43) Pub. Date: Jan. 22, 2004

(54) Aqueous dispersible steryl ester compositions

(76) Inventors: Melanie J. Goulson, Dayton, MN (US); Wade N. Schmelzer, St. Louis Park, MN (US); Chin Hong Paul Kim, Minnetonka, MN (US); Wen-Juin Shieh, Munster, IN (US)

Correspondence Address: Jeffrey J. Skelton, Esq., Cargill, Incorporated, Law Dept., P.O. Box 5624, Minneapolis, MN, 55440-5624, US

(21) Appl. No.: 10/411,423

(22) Filed: Apr. 10, 2003

Related U.S. Application Data
(60) Provisional application No. 60/371,767, filed on Apr. 10, 2002.

(51) Int. Cl. A61K 47/00; A61K 38/17

(52) U.S. Cl. 424/439; 514/2

(57) ABSTRACT

The present invention relates to water dispersible steryl ester compositions that include steryl esters, an emulsifier, and a protein or lipophilic starch stabilizer. Such compositions of the invention can be incorporated into aqueous foods and beverages.

### CROSS REFERENCE TO RELATED APPLICATIONS

This application claims priority from U.S. Provisional Application Serial No. 60/371,767, filed on Apr. 10, 2002.

### TECHNICAL FIELD

The present invention relates to the formulation and delivery of steryl esters. More specifically, it relates to water dispersible formulations of steryl esters.

### BACKGROUND

Coronary heart disease (CHD) is a common and serious form of cardiovascular disease that causes more deaths in the U.S. every year than any other disease. High serum cholesterol, and especially high levels of low density lipoprotein (LDL) cholesterol, are risk factors associated with CHD. Research has shown that esters of plant sterols or stanols (i.e., steryl esters) may lower total and LDL cholesterol and thereby reduce the risk of CHD.

One way that individuals can get more esters of sterols or stanols into their diets is by consuming fortified or “functional” foods. The FDA has authorized use of labeling health claims about the role of such esters in reducing the risk of coronary heart disease (CHD) for specific foods containing these substances. It advises that foods containing at least 0.65 g per serving of plant sterol esters, eaten twice a day with meals for a daily total intake of at least 1.3 g (or 1.7 g of stanol esters twice a day for a total of 3.4 g), as part of a diet low in saturated fat and cholesterol, may reduce the risk of heart disease.

Because steryl esters are fat soluble, hydrophobic ingredients, they typically have been utilized in fat based foods such as margarines, spreads, salad dressings, and mayonnaise. While this approach is a technical solution to formulating with these ingredients, it is contradictory and confusing to cholesterol-conscious consumers who are trying to reduce the amount of fat in their diets.

There is a need for technology to allow use of steryl esters in aqueous systems to facilitate healthier food choices. Further, the development of water dispersible steryl esters will increase the variety of foods consumers may choose from to achieve their twice daily servings of such esters.

### SUMMARY

The present invention is based on the discovery of formulations that allow steryl esters to be easily incorporated into aqueous foods and beverages and that are able to withstand conditions of heat, acid, and water hardness. Such compositions are powders or low viscosity, water-soluble fluids that are easily stirred into beverages or other non-fat foods. The compositions leave a clean mouthfeel without the waxy or mouthcoating characteristics that can be associated with sterols, stanols, steryl ester, and stanol ester fortified foods.

The compositions are complex oil-in-water emulsions that can be formulated to be bland, flavored, or sweetened. Advantageously, providing steryl esters in the form of the emulsified compositions improves the handling properties of the steryl esters. While steryl esters themselves are viscous, sticky, and difficult to measure and clean up, emulsions of steryl esters are very fluid (or powdered), easy to pump and measure, and readily water dispersible, properties that make handling and clean-up easier.

Any of the individual elements of the present invention may be comprised of mixtures of components that serve the function indicated. For example the protein identified in the claims of the present invention may be, but need not be, comprised of a mixture of protein components.

In one aspect, the invention features a composition that includes the following ingredients: 1 to 75 weight % (wt %) of a steryl ester; 0.3 to 10 wt % of a protein (e.g., a caseinate or sodium, calcium, or potassium caseinate salt); 0.1 to 5 wt % of an emulsifier (e.g., 0.1 to 2 wt %); 0.1 to 5 wt % of a buffer (e.g., dipotassium phosphate); and 0 to 98.5 wt % of a liquid. The protein can be present at 2 to 3 wt % of the composition. The composition further can include one or more additional components selected from the group consisting of bulking agents, thickeners, and fats and/or one or more additional components selected from the group consisting of flavorings, coloring agents, sweeteners, antioxidants, and flow agents. The fatty acid moiety of the steryl ester can be a blend of C16 to C18 carbon chains. The invention also features food compositions containing such steryl ester compositions (e.g., 0.5 to 60 wt % of such a steryl ester composition).

In some embodiments, the steryl ester can be 1 to 40 wt % of the composition and the liquid can be 20 to 98 wt % of the composition. In other embodiments, the steryl ester can be 1 to 75 wt % of the composition and the liquid can be 0 to 5 wt % of the composition.

A composition can include 1 to 10 wt % of a steryl ester; 0.5 to 1.5 wt % of a protein; 0.2 to 0.6 wt % of an emulsifier; 0.1 to 1.5 wt % of a buffer; 0.5 to 15 wt % of a bulking agent; 3 to 10 wt % of a fat; and 40 to 80 wt % of a liquid. In such a composition, the protein can be a caseinate, the emulsifier can include mono- and diglycerides or esters thereof, the buffer can be dipotassium phosphate, the bulking agent can be corn syrup solids, the fat can be a partially hydrogenated vegetable oil, and the liquid can be water.

In other embodiments, a composition includes 15 to 25 wt % of a steryl ester; 0.5 to 8 wt % of a protein; 0.2 to 1 wt % of an emulsifier; 0.1 to 1.5 wt % of a buffer; 0.5 to 35 wt % of a bulking agent; 0 to 4 wt % of a fat; and 35 to 80 wt % of a liquid. The invention also features food compositions containing such steryl ester compositions (e.g., 1 to 40 wt % of such a steryl ester composition).

A composition also can include about 5 to 15 wt % of a steryl ester, 1 to 5 wt % of a protein, 0.3 to 1.5 wt % of an emulsifier; 1 to 3 wt % of a buffer, 70 to 80 wt % of a bulking agent, and 5 to 10 wt % of a fat. The protein can be a caseinate, the emulsifier can include mono- and diglycerides or esters thereof, the buffer can be dipotassium phosphate, the bulking agent can be corn syrup solids, and the fat can be a partially hydrogenated vegetable oil. A food composition can include 20 to 40 wt % of such compositions.

In another aspect, the invention features a method of making an aqueous dispersible steryl ester composition, where the composition includes 1 to 75 wt % of a steryl ester, 0.3 to 10 wt % of a protein, 0.1 to 5 wt % of an emulsifier; 0.1 to 5 wt % of a buffer, and 0 to 98.5 wt % of a liquid. The method can include combining the steryl ester and emulsifier under conditions suitable to form a melt; combining the melt with the protein, wherein the protein is in an aqueous phase, to form an emulsion; and homogenizing the emulsion. The method further can include drying the emulsion to form a powdered product.

In yet another aspect, the invention features a composition that includes the following ingredients: 1 to 75 wt % of a steryl ester; 0.3 to 15 wt % of octenylsuccinate anhydride starch (OSAN/n-OSAN) (e.g., 1 to 3 wt %); 0.1 to 5 wt % of an emulsifier (e.g., 0.1 to 2 wt %); and 0 to 98.6 wt % of a liquid. Such a composition further can include one or more additional components selected from the group consisting of buffers, bulking agents, thickeners, and fats and/or one or more additional components selected from the group consisting of flavorings, coloring agents, sweeteners, antioxidants, and flow agents. The fatty acid moiety of the steryl ester can be a blend of C16 to C18 carbon chains. The steryl ester can be 1 to 40 wt % of the composition and the liquid can be 20 to 98 wt % of the composition. A composition also can include 1 to 75 wt % of a steryl ester 0 to 5 wt % of a liquid. The invention also features food compositions that include such steryl ester compositions (e.g., 0.5 to 60 wt %).

A composition can include 1 to 10 wt % of a steryl ester; 0.5 to 1.5 wt % of octenylsuccinate anhydride starch; 0.2 to 0.6 wt % of an emulsifier; 0.5 to 15 wt % of a bulking agent; 3 to 10 wt % of a fat; and 40 to 80 wt % of a liquid. The emulsifier can include mono- and diglycerides or esters thereof, the bulking agent can be corn syrup solids, the fat can be a partially hydrogenated vegetable oil, and the liquid can be water.

A composition also can include 15 to 25 wt % of a steryl ester; 0.5 to 8 wt % of octenylsuccinate anhydride starch; 0.2 to 1 wt % of an emulsifier; 0.1 to 1.5 wt % of a buffer; 0.5 to 35 wt % of a bulking agent; 0 to 4 wt % of a fat; and 35 to 80 wt % of a liquid.

The invention also features a method of making an aqueous dispersible steryl ester composition. The composition includes 1 to 75 wt % of a steryl ester, 0.3 to 15 wt % of octenylsuccinate anhydride starch, 0.1 to 5 wt % of an emulsifier; and 0 to 98.6 wt % of a liquid. The method includes combining the steryl ester and the emulsifier under conditions suitable to form a melt; combining the melt with the octenylsuccinate anhydride starch, wherein the starch is in an aqueous phase, to form an emulsion; and homogenizing the emulsion. The method further can include drying the emulsion to form a powdered product.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention features compositions that can be used to incorporate steryl esters into aqueous foods and beverages. Such compositions also are referred to as “premixes” throughout the application. In general, the premixes are complex oil-in-water emulsions that contain steryl esters and two types of emulsifiers, a protein or a lipophilic starch emulsifier (both referred to herein as a stabilizer) and a second, non-protein or non-starch emulsifier. Premixes also can include one or more of the following: buffers, bulking agents, thickeners, fats, flavorings, coloring agents, sweeteners, flow agents, and antioxidants. Compositions of the invention can be readily dispersed in a number of food products, including both cold, acidic beverages such as orange juice (especially when a lipophilic starch is used) and hot aqueous beverages such as tea and coffee. The versatility of the compositions can help consumers achieve their daily level of steryl esters via a simple cup of coffee or any number of other foods or beverages.

Steryl Ester

The term “steryl ester” as used herein means the fatty acid esters of specific plant phytosterols such as: sitosterol, campesterol, stigmasterol, brassicasterol, avenasterols, and diosgenin, or mixtures of specific sterols. The specific sterols or mixtures of sterols may be isolated from the following sources: oilseeds such as soybeans, canola seed, corn, sunflower, cottonseed, palm kernel, corn fiber, soy germ, shea nut, or peanut; tree sources such as tall oil (from pine trees), tall oil soap or tall oil pitch; other plant sources such as Mexican yam, olives, or sugar cane. In oilseeds, the most abundant phytosterols are sitosterol (˜52 to 89% of total sterols), campesterol (˜2 to 30% of total sterols), and stigmasterol (up to 26% of total sterols). The chemical structures of sitosterol, campesterol, and stigmasterol are provided below in formulas I and II.

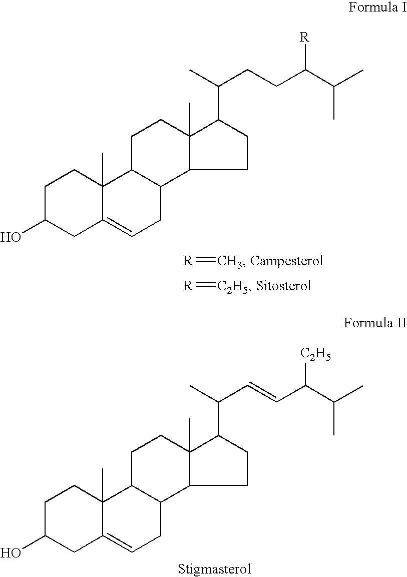

Also included within the definition of steryl esters are the esterified and hydrogenated forms of the above mentioned sterols (known in the art as stanols) including but not limited to sitostanol and campestanol. Further included within this definition are sterol ester derivatives such as ferulate esters, or succinate esters.

The fatty acid moiety of the steryl esters can have a carbon chain ranging from about 8 to about 20, with carbon chains of 16 to 18 being particularly useful. Blends of fatty acid moieties having C16 to C18 carbon chains can be isolated from vegetable oils, and in particular, from high oleic acid canola oil, high oleic soybean oil, and high oleic sunflower oil. Suitable canola oils are described, for example, in U.S. Pat. Nos. 5,861,187, 5,850,026, and 5,840,946. In addition, seeds of a canola plant line (line Q4275) that yield a canola oil having an oleic acid content greater than 80% have been deposited with the American Type Culture Collection under Accession No. 97569. High oleic sunflower oils having oleic acid contents of about 86% to about 92%, can be obtained from A. C. Humko, Memphis, Tenn. U.S. Pat. No. 4,627,192 describes a suitable high oleic acid sunflower oil. Known processes can be used to obtain fatty acids from vegetable oils.

The steryl esters described in the present invention are commercially available and well know in the art. Alternatively, steryl esters can be produced from phytosterols, which are typically recovered from deodorizer distillate produced during deodorization or refining of vegetable oils. Individual, purified phytosterols, e.g., purified sitosterol or purified stigmasterol, are available commercially, as are blends of sterols, e.g., soy sterol containing β-sitosterol, stigmasterol, and campesterol and others. Sterols can be obtained as free sterols or as sterol glycosides, in which a sugar moiety is attached to the hydroxyl group of the sterol, or as sterol esters, in which the hydroxyl group is attached to a fatty acid.

Steryl esters can be produced by transesterification, in which the alcohol moiety from a fatty acid ester, e.g., a fatty acid methyl ester, is displaced by another alcohol (in this case, free sterol). Free sterol and a fatty acid methyl ester can be reacted in the presence of base catalysts such as sodium hydroxide or sodium methoxide, an acid catalyst such as p-toluene sulfonic acid, metals such as BBr₃, Me₃SiI, Al₂O₃, Ti(OR)₄, DMAP, and n-BuLi, K t-butoxide, and enzymes such as lipases, esterases, and α-chymotrypsin. Preferably, the catalyst is food grade. Typically, free sterol is mixed with a molar excess of fatty acid esters (e.g., a 5 to 10% molar excess), and the mixture is heated until the sterols dissolve (approximately 115 to 140° C.) before addition of catalyst. The reaction can be stirred and heated under vacuum until completion, during which time methanol produced from the reaction can be condensed and collected. Alternatively, free sterols and free fatty acids can be directly esterified according to the methods described, for example, in U.S. Pat. No. 5,892,068.

Fatty acid methyl esters can be produced by either esterifying free fatty acids with methanol or transesterifying triacylglycerols with methanol. Such reactions can be performed batchwise or continuously. For example, batch transesterification of triacylglycerols with methanol can be performed with an excess of methanol and in the presence of a catalyst, e.g., an alkaline catalyst, under high pressure (9000 kPa) and high temperature (˜240° C.). See, Bailey's Industrial Oil & Fat Products: General Applications, Vol. 5, pp. 49-53, John Wiley & Sons, Inc., New York, N.Y. (1996). Similar conditions are used for continuous transesterification.

Steryl esters can be purified by solvent or aqueous extraction, bleaching and deodorization, or other known methods. For example, purified steryl esters can be obtained by aqueous extraction by suspending the reaction products in aqueous sodium bicarbonate (e.g., 1%), filtering the suspension to obtain purified steryl esters, and drying the purified steryl esters. Reaction products can be bleached using diatomaceous earth, bleaching clay, activated carbon, silica, or combinations thereof. Purity of steryl esters can be assessed by thin layer chromatography, gas chromatography (GC), or liquid chromatography (LC). LC is particularly useful.

Steryl Ester Compositions

Compositions of the invention include 1 to 75 weight percent (wt %) of a steryl ester, 0.3 to 15 wt % of a stabilizer, and 0 to 98.7 wt % of a liquid (e.g., water or milk), with the total wt % of a composition being 100. Ingredients within the compositions generally are considered food grade, generally recognized as safe (GRAS), and/or are U.S. Food and Drug Administration (FDA)-cleared. Typically, steryl esters are present in an amount that will provide at least 0.65 g of steryl esters per serving in the end product. As used herein, “stabilizer” refers to either a protein or a lipophilic starch. Suitable proteins are water soluble or dispersible. Caseinates, including the sodium, potassium, and calcium salts, are particularly useful. Other proteins, including other milk protein sources (e.g., non-fat milk solids or whey solids), soy protein, rice protein, wheat protein, oat protein, and mixtures also can be used (see, for example, U.S. Pat. No. 6,287,616). Protein hydrolysates (e.g., hydrolyzed soy or whey protein concentrates) also can be used. See U.S. Pat. No. 5,024,849. Generally, protein stabilizers are present at 0.3 to 10 wt % of the composition (e.g., 1 to 5 or 2 to 3 wt %).

Lipophilic starch refers to modified starch having both hydrophilic and hydrophobic substituents to give emulsion-stabilizing properties. Suitable lipophilic starches include starch alkenyl succinates, which are produced by treating starch with alkenyl succinic anhydride under controlled pH conditions. Octenylsuccinate anhydride starch (OSAN/n-OSAN), which has a hydrophilic carboxyl group and a hydrophobic C8 alkene chain, is particularly useful. OSAN/n-OSAN is available from Cargill, Inc. (EmCap Instant 12633) and National Starch (Purity Gum B. E., and N-creamer 46). Typically, lipophilic starch is present at about 0.3 to 15 wt % of the composition (e.g., 1 to 3, 3.5 to 7 wt % or 5 to 10 wt

The term “emulsifier” as used herein means long chain fatty acid esters including, but not limited to, distilled monoglycerides, mono- and diglycerides, and diacetyl tartartic acid esters of mono- and diglycerides (DATEM). Emulsifiers play an important role in formation of the emulsion and also act as dispersion aids in the end applications. Other emulsifiers that can be used include polysorbate 60, lecithin and modified lecithin, sodium stearyl lactylate, propylene glycol monostearate, succinylated mono- and diglycerides, acetylated mono- and diglycerides, propylene glycol mono- and diesters of fatty acids, polyglycerol esters of fatty acids, lactylic esters of fatty acids, glyceryl monostearate, and mixtures thereof. Emulsifiers can improve emulsification, stability, wetting dispersibility, as well as reduce the amount of necessary protein or lipophilic starch. Emulsifiers typically are present at 0.1 to 5 wt % (e.g., 0.1 to 2 wt %) of the composition.

In some embodiments, the compositions include a buffer. The term “buffer” as used herein means an organic or inorganic acid, base, or salt useful to control pH. Preferred buffers include, but are not limited to, phosphate buffers such as dipotassium phosphate, and citrate buffers such as sodium citrate. Buffers may be used alone or in combination to achieve desired results of protecting the stabilizer (e.g., protein or starch) against the effects of acid and hard water, which can result in a defect known as “feathering.” When present, buffers typically are 0.1 to 5 wt % of the composition and more particularly, 0.1 to 1.5 or 1 to 3 wt % of the composition.

Thickeners or hydrocolloids can be added to the composition of the present invention to contribute extra stabilization, viscosity, and body to the product. Carageenan is particularly useful, but other hydrocolloids, including alginates, xanthan gum, locust bean gum, gum arabic, guar gum, cellulose gum, and gum tragacanth, also can be used. Thickeners can be present at 0 to 5 wt % (e.g., 0.1 to 0.5 wt % or 0.1 to 0.3 wt %).

Sweeteners also can be included in a composition. Sweeteners can be used as bulking agents (i.e., to provide body or viscosity) or to impart a desired level of sweetness. Corn syrup solids are particularly useful and provide both body and moderate sweetness. Maltodextrins also can be used. Sugar or other nutritive sweeteners such as liquid corn sweeteners and dextrose can be used if a substantially sweeter product is desired. In some embodiments, a sugar substitute such as saccharine, aspartame, acesulfame potassium, or sucrolose can be used in a composition of the invention. Bulking agents that do not contribute to sweetness, e.g., starch or cellulose, also can be used.

A fat also can be employed in a composition of the present invention. As used herein, “fat” includes both liquid oils and solid or semi-solid fats. Suitable fats include partially or fully hydrogenated vegetable oils; tropical oils such as coconut, palm, and palm kernel oil; liquid oils such as soybean, canola, cottonseed, sunflower, safflower, corn, and their mid- and high-oleic counterparts; or any combination thereof. Partially hydrogenated vegetable oils are particularly useful. A fat can be present at 0 to 74 wt % of a composition of the invention, depending on the end application. Typically, a fat is present in a liquid composition at 1.5 to 7 wt % or 7 to 13 wt %. In powdered compositions, the fat content can range from 20 to 74 wt %. For example, in a coffee creamer, the fat content can be 20 to 35 wt %. In a powdered drink mix, the fat content can be 35 to 50 wt %. Other high fat compositions can have fat contents of 55 to 75 wt %. Although a composition contains fat, the small serving size (for example, 15 mL or less depending on steryl ester concentration) of the composition will have minimum impact in the end use (for example, a cup of coffee) and on an overall diet. It has been observed that, while not required, including a small portion of partially hydrogenated vegetable oil in the composition of the present invention aids in solubility of the steryl esters, and the formation and stability of the emulsion.

Additional ingredients can be included in compositions, including flavors (e.g., vanilla, hazelnut, Irish créme, mocha, almond, liqueurs, or chocolate); coloring agents; antioxidants to increase oxidative stability (e.g., ethoxyquin, vitamin E, butylated hydroxyanisole (BHA), butylated hydroxytoluene (BHT), tertiary butylhydroquinone (TBHQ), or ascorbyl palmitate); and flow agents (to prevent caking and clumping in dry products) such as sodium aluminosilicate or silicon dioxide.

Preparation of Steryl Ester Compositions

Compositions of the invention can be prepared by mixing and heating the steryl esters, the emulsifiers, and, optionally, a fat (e.g., a vegetable oil) to obtain a melt. Typically, the melt can be obtained by heating the components, with stirring, to approximately 155° F. The speed at which the melt is obtained can be accelerated by elevating the temperature. A skilled artisan will appreciate that the temperature required will vary depending upon the specific steryl esters and emulsifiers utilized.

The aqueous phase can be prepared by dissolving a buffer and stabilizer (i.e., protein or lipophilic starch) in water and heating the solution to a temperature close to that of the steryl ester/emulsifier melt. The aqueous phase then can be slowly added to the melt while blending with high shear to obtain an emulsion. Additional desired components (e.g., bulking agents, flavorings, fats, coloring agents, flow agents, or antioxidants) can be added with mixing to obtain a uniform mixture. The mixture then can be pasteurized or otherwise heat processed using known techniques. To reduce particle size of the fat droplets, a two-stage homogenization can be done (e.g., at 2500 psi and 500 psi). The resulting homogenized product can be packaged to produce a liquid premix or spray dried to produce a powdered premix product. The powdered form may be agglomerated to instantize the product and improve dispersibility when it is stirred into the aqueous end application.

Formulating Compositions of the Invention

Compositions of the invention can be liquids, gels, or powders, and can be formulated such that the steryl esters are either dilute or concentrated, depending on the desired end use. Typically, liquid compositions of the invention include from 1 to 40 wt % of a steryl ester, 0.3 to 15 wt % of a stabilizer, 0.1 to 5 wt % of an emulsifier, and from 20 to 98.6 wt % of a liquid. For example, a liquid composition containing a relatively low amount of steryl esters can include 1 to 10 wt % of a steryl ester, 0.5 to 1.5 wt % of a stabilizer (e.g., a caseinate such as sodium caseinate), 0.2 to 0.6 wt % of an emulsifier (e.g., mono- and diglycerides and/or diacetyl tartartic acid esters thereof); 0.1 to 1.5 wt % of a buffer (e.g., dipotassium phosphate), 0.5 to 15 wt % of a bulking agent (e.g., corn syrup solids); 3 to 10 wt % of a fat (e.g., partially hydrogenated vegetable oil), and 40 to 80 wt % of a liquid (e.g., water). Such a composition is a stable emulsion that is fluid and easily dispersible and can be used as a creamer for tea or coffee. Such compositions further can include sweetener and flavorings. For example, 25 to 30 wt % of a sweetener (e.g., sugar), and 0.1 to 2 wt % of a flavoring (e.g., French vanilla) can be added.

In other embodiments, a liquid composition can be used as a beverage premix emulsion and contain a more concentrated amount of steryl esters. For example, a liquid composition can include 15 to 25 wt % of a steryl ester, 0.5 to 8 wt % of a stabilizer (e.g., a caseinate such as sodium caseinate or a lipophilic starch), 0.2 to 1 wt % of an emulsifier (e.g., mono- and diglycerides and/or diacetyl tartartic acid esters thereof); 0.1 to 1.5 wt % of a buffer (e.g., dipotassium phosphate), 0.5 to 35 wt % of a bulking agent (e.g., corn syrup solids); 0 to 4 wt % of a fat (e.g., partially hydrogenated vegetable oil), and 35 to 80 wt % of a liquid (e.g., water).

In other embodiments, the composition can be dry (e.g., powdered) and include 1 to 75 wt % of a steryl ester and 0 to 5 wt % of a liquid. It is noted that “dry” material may contain residual levels of liquid. Powdered forms of the composition can be used in powdered drink mixes with relatively larger serving sizes (e.g., cappuccino mix or hot chocolate mix). For example, a powdered composition can include about 5 to 15 wt % of a steryl ester, 1 to 5 wt % of a stabilizer (e.g., a caseinate such as sodium caseinate), 0.3 to 1.5 wt % of an emulsifier (e.g., mono- and diglycerides and/or diacetyl tartartic acid esters thereof), 1 to 3 wt % of a buffer (e.g., dipotassium phosphate), 70 to 80 wt % of a bulking agent (e.g., corn syrup solids), and 5 to 10 wt % of a fat (e.g., partially hydrogenated vegetable oil). See, for example, the compositions of Examples 6 and 7. In such a composition, the water for the oil-in-water emulsion is provided by the water in which the buffer and stabilizer are dissolved or dispersed.

Compositions containing a higher concentration of steryl esters can be produced by concentrating a liquid composition using evaporative or drying techniques such as spray drying drum-drying, or tray drying, or any combination thereof. As a result, the amount of liquid in the composition can be reduced to a desired end point. In some embodiments, it may be desirable to produce compositions having a gel-like consistency and a liquid content of about 5 to 20 wt %.

In other embodiments, it may be desired to produce powdered compositions. For example, a liquid composition containing 20 to 23 wt % of steryl esters, 0.5 to 7 wt % of a stabilizer (e.g., 0.5 to 2 wt % of a protein such as sodium caseinate or 4 to 7 wt % of a lipophilic starch), 0.3 to 1 wt % of an emulsifier, 1 to 2 wt % of a buffer, 3 to 40 wt % of a bulking agent (e.g., corn syrup solids), 0 to 10 wt % of a fat, and 35 to 45 wt % of a liquid (e.g., water), can be spray dried to obtain an easily dispersible powder. After drying, such compositions contain approximately 30 to 35 wt % of steryl esters. See, for example, the compositions of Examples 3, 5, and 8-10. When such compositions contain a lipophilic starch, it is particularly useful for adding to orange juice, powdered fruit drinks such as Crystal Light®, or other acidic beverages to provide the desired serving of steryl esters.

Compositions of the invention can be incorporated into a variety of food or beverages. For example, liquid or powdered compositions can be added to coffee and tea beverages (i.e., as a coffee creamer), as well as milk, yogurt and yogurt drinks, cocoa, instant breakfast drinks, soy drinks, juice drinks and smoothies, and meal replacement beverages. As described herein, a water dispersible steryl ester composition of the invention containing 35 wt % of steryl esters had superior whitening properties than a commercially available non-dairy creamer product. In addition, compositions can be added to milk or another liquid that is subsequently used in the preparation of another food such as breakfast cereal (e.g., instant oatmeal), scrambled egg whites, mashed potatoes, or pudding. Compositions also can be used in the preparation of soups, sauces, dressings, mousse, desserts, and toppings. Formulations can be assessed for palatability and mouthfeel using a panel of trained taste testers according to known techniques.

The amount of steryl ester composition that is incorporated into the product (e.g., powdered drink mix, liquid coffee creamer, or beverage premix emulsion) will depend on the end use and the desired concentration of steryl esters in the product. Typically, steryl ester compositions of the invention represent 0.5 to 60 wt % (e.g., 1 to 40 wt %) of the end product. For example, as described in Example 7, a low fat instant cappuccino mix can be produced that will deliver 0.65 g of steryl esters per serving. Such a mix typically contains 50 to 60 wt % sugar, 25 to 35 wt % of a dilute steryl ester composition of the invention, 10 to 15 wt % nonfat dry milk, 1 to 2 wt % instant coffee, carboxymethylcellulose (CMC) 0.1 to 1 wt %, 0.1 to 1 wt % of a flow agent, 0.1 to 0.75 wt % salt, and 0.1 to 1.0 wt % of a flavoring agent. Steryl ester compositions stabilized by a lipophilic starch can be stirred into a powdered low calorie fruit drink mix to provide the desired serving of steryl esters. See Example 9.

The invention will be further described in the following examples, which does not limit the scope of the invention described in the claims.

### EXAMPLES

### Example 1

### Unsweetened Steryl Ester Premix for Delivering 0.65 g Steryl Esters per 15 mL Serving.

An unsweetened steryl ester premix was prepared using the ingredients listed in Table 1.

TABLE 1

| Ingredient | % (by weight) |
|---|---|
| Steryl Esters (Cargill Incorporated) | 5.00 |
| Partially Hydrogenated Soybean Oil (Cargill S700, IV =) | 5.00 |
| Distilled Monoglycerides (Danisco DIMODAN OK) | 0.20 |
| DATEM (Danisco PANODAN FDPK) | 0.20 |
| Water | 78.35 |
| Dipotassium Phosphate (Astaris) | 0.20 |
| Sodium Caseinate (New Zealand Milk Products Alanate | 1.00 |
| 110) | |
| Carageenan (Danisco CL220) | 0.10 |
| 25 DE Corn Syrup Solids (Cerestar Dry MD 01915) | 10.00 |
| Total | 100.00 |

Procedure:

1. The steryl esters, soybean oil, and emulsifiers (distilled monoglycerides and DATEM) were combined and heated with mixing to 155° F.

2. Dipotassium phosphate was dissolved in the water and heated to 120° F.

3. The sodium caseinate and carrageenan were pre-mixed then slowly added to the dipotassium phosphate solution with mixing to prevent clumping. The mixture was heated to 150° F. and held for 10 minutes while mixing to achieve complete solubilization.

4. The water phase from step 3 was slowly poured into the steryl ester phase while blending with high shear to form an emulsion.

5. Corn syrup solids were gradually added to the emulsion with vigorous mixing until a uniform mixture was obtained.

6. The mixture was batch pasteurized at 165° F. for 15 minutes.

7. The pasteurized mixture was homogenized in a two-stage process at 2500 psi and 500 psi.

The resulting product was a very fluid, bland tasting, stable emulsion that was easily dispersible.

### Example 2

### Concentrated, Unsweetened Steryl Ester Premix

A concentrated unsweetened steryl ester premix was prepared using the ingredients listed in Table 2. The premix was prepared as described in steps 1 to 5 of Example 1. This formula yields a slightly thicker, smooth, flowable, stable emulsion that was easily dispersible. Approximately 3.5 g of premix delivers 0.65 g steryl esters.

TABLE 2

| Ingredient | % (by weight) |
|---|---|
| Steryl Esters (Cargill) | 19.00 |
| Partially Hydrogenated Soybean Oil (Cargill S700, IV =) | 2.50 |
| Distilled Monoglycerides (Danisco DIMODAN OK) | 0.20 |
| DATEM (Danisco PANODAN FDPK) | 0.20 |
| Water | 74.85 |
| Dipotassium Phosphate (Astaris) | 1.20 |
| Sodium Caseinate (New Zealand Milk Products Alanate | 1.00 |
| 110) | |
| Carageenan (Danisco CL220) | 0.05 |
| 25 DE Corn Syrup Solids (Cerestar Dry MD 01915) | 1.00 |
| Total | 100.00 |

### Example 3

### Unsweetened Powdered Steryl Ester Premix.

An unsweetened powdered steryl ester premix was prepared by following steps 1 through 5 of Example 1. The mixture then was homogenized in a two-stage process at 3000 psi and 500 psi. The emulsion was spray dried using standard techniques to produce a fine, white, easily dispersible powder.

TABLE 3

| Ingredient | % (by weight) |
|---|---|
| Steryl Esters (Cargill) | 21.00 |
| Distilled Monoglycerides (Danisco DIMODAN OK) | 0.20 |
| DATEM (Danisco PANODAN FDPK) | 0.20 |
| Water | 40.10 |
| Dipotassium Phosphate (Astaris) | 1.20 |
| Sodium Caseinate (New Zealand Milk Products Alanate | 1.80 |
| 110) | |
| 25 DE Corn Syrup Solids (Cerestar Dry MD 01915) | 35.50 |
| Total | 100.00 |

### Example 4

### Sweetened and Flavored Steryl Ester Premix:

A sweetened and flavored steryl ester premix was prepared using the ingredients in Table 4.

TABLE 4

| Ingredient | % (by weight) |
|---|---|
| Partially Hydrogenated Soybean Oil (Cargill S700, IV =) | 8.75 |
| Steryl Esters (Cargill) | 4.70 |
| Distilled Monoglycerides (Danisco DIMODAN OK) | 0.20 |
| DATEM (Danisco PANODAN FDPK) | 0.20 |
| Water | 49.847 |
| Dipotassium Phosphate (Astaris) | 1.20 |
| Sodium Caseinate (New Zealand Milk Products Alanate | 1.00 |
| 110) | |
| Carageenan (Danisco CL220) | 0.10 |
| 25 DE Corn Syrup Solids (Cerestar Dry MD 01915) | 3.00 |
| Sugar | 30.00 |
| Salt | 0.003 |
| French Vanilla Flavoring | 1.00 |
| Total | 100.00 |

Procedure:

1. The steryl esters, soybean oil, and emulsifiers (distilled monoglycerides and DATEM) were combined with mixing and heated to 155° F. to form a melt.

2. Dipotassium phosphate was dissolved in water and heated to 120° F.

3. The sodium caseinate, carrageenan, sugar, and salt were premixed and slowly added to the dipotassium phosphate solution with mixing to prevent clumping. The resulting aqueous phase was heated to 150° F. and held for 10 minutes while mixing to solubilize.

4. The water phase was slowly poured into the steryl ester phase while stirring to form an emulsion.

5. The flavoring and corn syrup solids were gradually added to the emulsion with vigorous mixing until a uniform mixture was obtained.

6. The formulation was blended in a blender with high shear for 1 minute.

This product was a very fluid, sweet vanilla tasting, stable emulsion that was easily dispersible.

### Example 5

### Coffee Sweetened, Whitened, and Fortified with a Steryl Ester Premix

A 15 mL serving of a liquid steryl ester premix (as described in Examples 1 and 4) was poured into a freshly brewed cup (6 oz, 180 mL) of hot coffee and stirred with a spoon to disperse. The result was a very pleasant tasting, whitened coffee with creamy mouthfeel, reduced bitterness, and a healthful dose of steryl esters (0.65 g). The creamer remained dispersed in the coffee for over an hour (until the coffee had cooled to room temperature) with no apparent feathering or oiling out.

### Example 6

### Water Dispersible Steryl Ester for Powdered Drink Mix Applications.

In this example, steryl esters are used in combination with partially hydrogenated vegetable oil (9.00% and 7.76%, respectively) to produce a water dispersible powder that can be used in powdered drink mixes such instant cappuccino.

TABLE 5

| Ingredient | % (by weight) |
|---|---|
| Dipotassium Phosphate | 2.00 |
| Sodium Caseinate | 3.00 |
| Corn Syrup Solids | 77.21 |
| Steryl Esters | 9.00 |
| Partially Hydrogenated Vegetable Oil | 7.76 |
| Mono- and Diglycerides | 0.70 |
| DATEM | 0.33 |
| Total | 100.00 |

Procedure:

1. The steryl ester, vegetable oil, and emulsifiers (mono- and diglycerides and DATEM) are combined together and are heated with mixing to 155° F.

2. The dipotassium phosphate is dissolved in the water and is heated to 120° F.

3. The caseinate is slowly added to the dipotassium phosphate solution with mixing. Heat to 150° F. and hold for 10 minutes for complete solubilization.

4. The water phase from step 3 is slowly poured into the steryl ester phase while blending with high shear to form an emulsion.

5. The corn syrup solids are gradually added to the emulsion with mixing until dissolved.

6. The emulsion is batch pasteurized at 165° F. for 15 minutes.

7. The pasteurized emulsion is homogenized in a two-stage process at 2500/500 psi.

8. The homogenized emulsion is spray dried with an inlet temperature of 400° F. and outlet temperature of 200° F.

### Example 7

### Low Fat Instant Cappuccino Mix Containing Water Dispersible Steryl Esters.

A low fat instant cappuccino mix is prepared by dry blending all ingredients listed in the following Table. The mix, which contains 29% water dispersible steryl esters, will deliver 0.65 g of steryl esters per serving. To make a cup of cappuccino, add 6 oz (180 g) of hot water to 25 g of cappuccino mix and stir.

TABLE 6

| Ingredient | % (by weight) |
|---|---|
| Sugar | 56.50 |
| Water Dispersible Steryl Esters | 29.00 |
| (9% steryl esters) | |
| Nonfat Dry Milk | 11.00 |
| Instant Coffee | 1.50 |
| CMC, high viscosity | 0.50 |
| Salt | 0.50 |
| Flavor | 0.50 |
| Flow Agent | 0.50 |
| Total | 100.00 |

### Example 8

### Water Dispersible Steryl Ester Composition for Powdered Drink Mix Applications:

A water dispersible steryl ester composition was made using the ingredients in Table 7. In this formulation, lipophilic starch was used in place of sodium caseinate. The spray-dried product contained 35 wt % of steryl esters. This product is relatively concentrated and particularly useful in applications with a small serving size. The starch stabilizer shows improved acid stability.

TABLE 7

| Ingredient | % (by weight) |
|---|---|
| Water | 40.10 |
| Potassium Diphosphate | 1.20 |
| EmCap Instant 12633 Lipophilic Starch | 5.60 |
| 25 DE Corn Syrup Solids | 31.35 |
| Steryl Esters | 21.00 |
| Monoglycerides | 0.50 |
| DATEM | 0.25 |
| Total | 100.00 |

Procedure:

1. Steryl esters and emulsifiers were combined together with mixing and heated to 155° F.

2. Dipotassium phosphate was dissolved in the water and heated to 120° F.

3. The starch was slowly added to the dipotassium phosphate solution with mixing to form the water phase.

4. The water phase was slowly poured into the steryl ester phase while blending with high shear to form an emulsion.

5. The corn syrup solids were gradually added to the emulsion with mixing until dissolved.

6. The emulsion was pasteurized at 165° F. for 15 minutes.

7. The emulsion was homogenized in a two-stage process at 2500/500 psi.

8. The homogenized emulsion was spray dried with an inlet temperature of 400° F. and an outlet temperature of 200° F.

### Example 9

### Low Calorie Powdered Fruit Drink Containing Water Dispersible Steryl Esters

The following product contains 40% of the composition described in Example 8 and will deliver 0.65 g steryl esters per serving. To make a glass of fruit drink, add 8 oz (240 g) of cold water to 5 g of the fruit drink powder and stir.

TABLE 8

| Ingredient | % (by weight) |
|---|---|
| Powdered low calorie fruit drink | 60.0 |
| Water Dispersible Steryl Esters | 40.0 |
| Total | 100.0 |

### Example 10

### Orange Juice Supplemented with Water Dispersible Steryl Esters

The following formula describes use of the composition of Example 8 in an orange juice application. The formula contains 0.65 g steryl esters per 8 oz (240 g) serving of juice. The lipophilic starch stabilized form of the invention can be easily stirred into a cold, acidic beverage and disperses well. To make a glass of steryl ester fortified orange juice, add 1 teaspoon (2 g) of water dispersible steryl esters (e.g., from Example 8) to an 8 oz glass of orange juice and stir.

TABLE 9

| Ingredient | % (by weight) |
|---|---|
| Single Strength (ready to drink) Orange Juice | 99.1 |
| Water Dispersible Steryl Esters | 0.9 |
| Total | 100.0 |

### Other Embodiments

The above specification, examples and data provide a complete description of the manufacture and use of the product of the invention. Since many embodiments of the invention can be made without departing from the spirit and scope of the invention, the invention resides in the claims hereinafter appended.

What is claimed:

1. A composition comprising the following ingredients:
a) 1 to 75 weight % of a steryl ester;
b) 0.3 to 10 weight % of a protein;
c) 0.1 to 5 weight % of an emulsifier;
d) 0.1 to 5 weight % of a buffer; and
e) 0 to 98.5 weight % of a liquid.

2. The composition of claim 1, wherein said protein is a caseinate.

3. The composition of claim 2, wherein said caseinate is present at 2 to 3 weight % of said composition.

4. The composition of claim 1, wherein said caseinate is a sodium, calcium, or potassium caseinate salt.

5. The composition of claim 1, wherein said buffer is dipotassium phosphate.

6. The composition of claim 1, said composition further comprising one or more additional components selected from the group consisting of bulking agents, thickeners, and fats.

7. The composition of claim 1, said composition further comprising one or more additional components selected from the group consisting of flavorings, coloring agents, sweeteners, antioxidants, and flow agents.

8. The composition of claim 1, wherein said emulsifier is 0.1 to 2 weight % of said composition.

9. The composition of claim 1, wherein the fatty acid moiety of said steryl ester is a blend of C16 to C18 carbon chains.

10. The composition of claim 1, wherein said steryl ester is 1 to 40 weight % of said composition and said liquid is 20 to 98 weight % of said composition.

11. The composition of claim 1, wherein said steryl ester is 1 to 75 weight % of said composition and said liquid is 0 to 5 weight % of said composition.

12. A food composition comprising 0.5 to 60 weight % of said composition of claim 1.

13. The composition of claim 7, said composition comprising 1 to 10 weight % of said steryl ester; 0.5 to 1.5 weight % of said protein; 0.2 to 0.6 weight % of said emulsifier; 0.1 to 1.5 weight % of a buffer; 0.5 to 15 weight % of a bulking agent; 3 to 10 weight % of a fat; and 40 to 80 weight % of said liquid.

14. The composition of claim 13, wherein said protein is a caseinate, said emulsifier comprises mono- and diglycerides or esters thereof, said buffer is dipotassium phosphate, said bulking agent is corn syrup solids, said fat is a partially hydrogenated vegetable oil, and said liquid is water.

15. The composition of claim 7, said composition comprising 15 to 25 weight % of a steryl ester; 0.5 to 8 weight % of a protein; 0.2 to 1 weight % of an emulsifier; 0.1 to 1.5 weight % of said buffer; 0.5 to 35 weight % of a bulking agent; 0 to 4 weight % of a fat; and 35 to 80 weight % of a liquid.

16. A food composition comprising 1 to 40 weight % of said composition of claim 15.

17. The composition of claim 7, said composition comprising about 5 to 15 weight % of said steryl ester, 1 to 5 weight % of said stabilizer, 0.3 to 1.5 weight % of said emulsifier; 1 to 3 weight % of said buffer, 70 to 80 weight % of a bulking agent, and 5 to 10 weight % of a fat.

18. The composition of claim 17, wherein said protein is a caseinate, said emulsifier comprises mono- and diglycerides or esters thereof, said buffer is dipotassium phosphate, said bulking agent is corn syrup solids, and said fat is a partially hydrogenated vegetable oil.

19. A food composition comprising 20 to 40 weight % of said composition of claim 18.

20. A method of making an aqueous dispersible steryl ester composition, said composition comprising 1 to 75 weight % of a steryl ester, 0.3 to 10 weight % of a protein, 0.1 to 5 weight % of an emulsifier; 0.1 to 5 weight % of a buffer, and 0 to 98.5 weight % of a liquid, said method comprising
a) combining said steryl ester and said emulsifier under conditions suitable to form a melt;
b) combining said melt with said protein, wherein said protein is in an aqueous phase, to form an emulsion; and
c) homogenizing said emulsion.

21. The method of claim 20, said method further comprising drying said emulsion to form a powdered product.

22. A composition comprising the following ingredients:
a) 1 to 75 weight % of a steryl ester;
b) 0.3 to 15 weight % of octenylsuccinate anhydride starch;
c) 0.1 to 5 weight % of an emulsifier; and
d) 0 to 98.6 weight % of a liquid.

23. The composition of claim 22, wherein octenylsuccinate anhydride starch is present at 1 to 3 weight %.

24. The composition of claim 22, said composition further comprising one or more additional components selected from the group consisting of buffers, bulking agents, thickeners, and fats.

25. The composition of claim 22, said composition further comprising one or more additional components selected from the group consisting of flavorings, coloring agents, sweeteners, antioxidants, and flow agents.

26. The composition of claim 22, wherein said emulsifier is 0.1 to 2 weight % of said composition.

27. The composition of claim 22, wherein the fatty acid moiety of said steryl ester is a blend of C16 to C18 carbon chains.

28. The composition of claim 22, wherein said steryl ester is 1 to 40 weight % of said composition and said liquid is 20 to 98 weight % of said composition.

29. The composition of claim 22, wherein said steryl ester is 1 to 75 weight % of said composition and said liquid is 0 to 5 weight % of said composition.

30. A food composition comprising 0.5 to 60 weight % of said composition of claim 22.

31. The composition of claim 25, said composition comprising 1 to 10 weight % of said steryl ester; 0.5 to 1.5 weight % of octenylsuccinate anhydride starch; 0.2 to 0.6 weight % of said emulsifier; 0.5 to 15 weight % of a bulking agent; 3 to 10 weight % of a fat; and 40 to 80 weight % of said liquid.

32. The composition of claim 31, wherein said emulsifier comprises mono- and diglycerides or esters thereof, said bulking agent is corn syrup solids, said fat is a partially hydrogenated vegetable oil, and said liquid is water.

33. The composition of claim 25, said composition comprising 15 to 25 weight % of said steryl ester; 0.5 to 8 weight % of octenylsuccinate anhydride starch; 0.2 to 1 weight % of said emulsifier; 0.1 to 1.5 weight % of a buffer; 0.5 to 35 weight % of a bulking agent; 0 to 4 weight % of a fat; and 35 to 80 weight % of said liquid.

34. A method of making an aqueous dispersible steryl ester composition, said composition comprising 1 to 75 weight % of a steryl ester, 0.3 to 15 weight % of octenylsuccinate anhydride starch, 0.1 to 5 weight % of an emulsifier; and 0 to 98.6 weight % of a liquid, said method comprising
a) combining said steryl ester and said emulsifier under conditions suitable to form a melt;
b) combining said melt with said octenylsuccinate anhydride starch, wherein said octenylsuccinate anhydride starch is in an aqueous phase, to form an emulsion; and
c) homogenizing said emulsion.

35. The method of claim 34, said method further comprising drying said emulsion to form a powdered product.
